(19) United States
(12) Patent Application Publication
(10) Pub. No.: US 2009/0111993 A1
(43) Pub. Date: Apr. 30, 2009

(54) Process for Preparing Beta-(Fluorophenyl)-Propanoate Ester Derivatives

(75) Inventors: John Oldfield, Macclesfield (GB); Ian Patel, Bristol (GB); Andrew Williams, Macclesfield (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 12/093,664

(22) PCT Filed: Nov. 13, 2006
(86) PCT No.: PCT/GB2006/004205

§ 371 Date: May 14, 2008

(30) Foreign Application Priority Data
Nov. 16, 2005 (SE) 0502515-0

(51) Int. Cl. C07D 211/34

(52) U.S. Cl. 546/238

(57) ABSTRACT

A process for preparing a compound of formula (I) comprising reacting a compound of formula (II) with a fluorinated boron species of formula (III) in the presence of: an alcohol; a rhodium (I) pre-catalyst species; a suitable ligand that binds to the rhodium (I) pre-catalyst species to form a catalyst complex; a base; and, a suitable solvent; the process being carried out at a temperature in the range 40 to 110° C. The compounds of formula (I) are useful in the preparation of pharmaceutically active compounds.

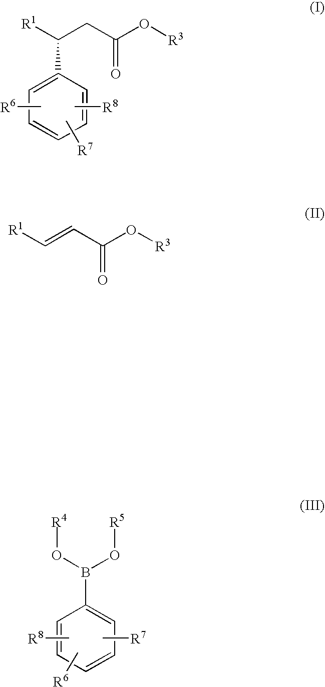

The present invention relates to a process for preparing asymmetric β-(fluorophenyl)-propanoate ester derivatives by reacting a fluorinated-phenyl-boronic acid or ester species with an α,β-unsaturated propenoate ester species in the presence of a chiral rhodium (I) catalyst complex and a base. β-(Fluorophenyl)-propanoate derivatives are useful as intermediates in the preparation of pharmaceuticals. (See, for example, WO 2004/056773 and WO 2005/009959.)

The asymmetric 1,4-addition of boronic acids and esters (together: boron species) to α,β-unsaturated ketones and α,β-unsaturated acids is disclosed in Synlett. (2002) 1791-4; Org. Lett. (2003) 5 681-4; J. Am. Chem. Soc. (2002) 124 5052-8; J. Org. Chem. (2000) 65 5951-5; J. Am. Chem. Soc. (1998) 124 5579-80; J. Org. Chem. (2001) 66 6852-6; Tetrahedron: Asymmetry (1999) 10 4047-56; J. Org. Chem. (2001) 66 8944-6; WO 2004/056773; and, WO 2005/009959.

In all these publications an excess of the boron species is used (2 equivalents or more—sometimes as much as 10 equivalents) due to rhodium-mediated protodeboronation of the boron species under the process conditions. It is postulated that the mechanism of protodeboronation involves an aryl-rhodium species (formed during the process) being intercepted by a molecule of water or alcohol and forming a corresponding arene. Alternatively, it is postulated that the productive forward reaction involves the same aryl-rhodium species complexing and then oxidatively inserting into the propenoate ester derivative.

An inorganic base, in solid form, is used in the processes disclosed above and, therefore, the reaction mixture of these processes is heterogeneous.

The problem of fast protodeboronation of fluorinated boron species is identified in J. Org. Chem. (2001) 66 6852-6 and Org. Lett. (2003) 5 (5) 681-4. This is a significant problem because of the high cost of fluorinated boron species. The present invention overcomes this problem by using up to 2 equivalents of an alcohol in place of water, thus allowing a smaller amount of fluorinated boron species to be used in the process thereby reducing the cost of goods of the process. Although alcohols can also be involved with the degradation of the boron species, the rate of degradation is considerably slower in the presence of an alcohol, relative to the rate with an equivalent presence of water.

As has already been said, the reaction mixture of the above processes is heterogeneous. A further advantage of using an alcohol instead of water is that, when using water, the particles of base agglomerate thus reducing the surface area of the base available for reaction, and creating a less efficient mixing system, thereby significantly impeding the progress of the reaction. This is of particular importance when working on large scale. When an alcohol is used instead of water, the base stays as finely divided particles (that is, there is no agglomeration) and an effective mixing system is maintained. The use of the alcohol therefore results in an acceptable reaction rate and a more robust and reliably scalable process.

Thus, the present invention provides a process for preparing a compound of formula (I):

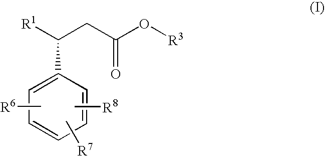

wherein R¹ is N-substituted piperidin-4-yl or optionally substituted phenyl; R³ is C₁₋₆ alkyl, optionally substituted phenyl or optionally substituted phenyl(C₁₋₄ alkyl); R⁶ is fluoro; and R⁷ and R⁸ are, independently, hydrogen or fluoro; the process comprising reacting a compound of formula (II):

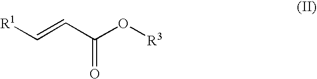

with 1 to 2 (for example 1 to 1.5) molar equivalents of a fluorinated boron species of formula (III):

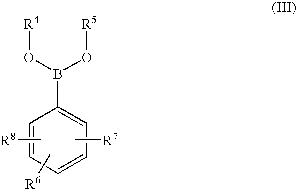

wherein R⁴ and R⁵ are, independently, hydrogen, C₁₋₆ alkyl, phenyl or phenyl(C₁₋₄ alkyl); or
R⁴ and R⁵ join to form a ring;
in the presence of:
0.8 to 1.5 molar equivalents of an alcohol;
a rhodium (I) pre-catalyst species;
a suitable ligand that binds to the rhodium (I) pre-catalyst species to form a catalyst complex;
a base; and,
a suitable solvent;
the process being carried out at a temperature in the range 40 to 110° C.

In another aspect when R¹ is optionally substituted phenyl it is, for example, phenyl optionally substituted by halo, S(O)₂(C₁₋₄ alkyl), S(O)₂(C₁₋₄ haloalkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C₁₋₄ haloalkyl, C₁₋₄ haloalkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl) or C(O)(C₁₋₄ haloalkyl).

In yet another aspect when R¹ is optionally substituted phenyl it is, for example, phenyl singly substituted (for example in the 4-position) by halo, S(O)₂(C₁₋₄ alkyl), S(O)₂(C₁₋₄ haloalkyl), C(O)(C₁₋₄ alkyl) or C(O)(C₁₋₄ haloalkyl).

In a further aspect R¹ is 4-substituted phenyl wherein the substituent is S(O)₂(C₁₋₄ alkyl) (such as S(O)₂CH₃).

N-substituted piperidin-4-yl is, for example, piperidin-4-yl with C₁₋₄ alkyl, S(O)₂(C₁₋₄ alkyl), S(O)₂(C₁₋₄ haloalkyl), C(O)(C₁₋₄ alkyl) or C(O)(C₁₋₄ haloalkyl) on the ring nitrogen. In a still further aspect N-substituted piperidin-4-yl is, for example, piperidin-4-yl with S(O)₂(C₁₋₄ alkyl) (such as S(O)₂CH₃) on the ring nitrogen.

In a further aspect R³ is ethyl, iso-propyl or tert-butyl.

When R⁴ and R⁵ join to form a ring they join, for example, to form (CR′R″)ₙ where n is 2, 3, 4, 5 or 6; and R′ and R″ are, independently, hydrogen or C₁₋₄ alkyl, and R′ and R″ can be different on different carbons. Thus when R⁴ and R⁵ join to form a ring the carbon chain formed by R⁴ and R⁵ is, for example, CH₂—C(CH₃)₂—CH₂ (neopentyl) or C(CH₃)₂—C(CH₃)₂ (pinacol).

In another aspect R⁶ is 3-fluoro; and R⁷ and R⁸ are, independently, hydrogen or fluoro (for example R⁷ is 5-fluoro or hydrogen, and R⁸ is hydrogen).

In yet another aspect R⁴ and R⁵ are, for example, hydrogen, C₁₋₄ alkyl or join to form (CR′R″)ₙ where n is 2, 3 or 4; and R′ and R″ are, independently, hydrogen or C₁₋₄ alkyl, and R′ and R″ can be different on different carbons. For example R⁴ and R⁵ are, independently, hydrogen, methyl or ethyl, or, when R⁴ and R⁵ join to form a ring the carbon chain formed by R⁴ and R⁵ is, for example, CH₂—C(CH₃)₂—CH₂ (neopentyl) or C(CH₃)₂—C(CH₃)₂ (pinacol).

In a further aspect the present invention provides a process wherein R⁷ is F and R⁸ is H.

In a still further aspect the present invention provides a process wherein R⁶ is 3-F, R⁷ is 5-F and R⁸ is H.

In another aspect the present invention provides a process wherein R¹ is N—(SO₂CH₃)piperidin-4-yl or 4-(SO₂CH₃)phenyl.

An alcohol is, for example, a C₁₋₁₀ aliphatic straight or branched chain acyclic alcohol (for example ethanol, propanol, iso-propanol, iso-butanol, sec-butanol or tert-butanol) or a C₃₋₁₀ cyclic alcohol (for example cyclohexanol, cyclobutanol or cyclopentanol).

A base is, for example, a phosphate, carbonate or bicarbonate of an alkali metal or alkaline earth metal, such as sodium carbonate, potassium carbonate or potassium phosphate.

A rhodium (I) pre-catalyst species is, for example, acetylacetobis[ethylene]rhodium (I), [Rh(COD)Cl]₂ or [Rh(COD)(MeCN)₂]BF₄ (where COD is cyclooctadiene).

Suitable ligands that bind to the rhodium (I) pre-catalyst species to form a catalyst complex are, for example, (R)-BINAP, (R)-tol-BINAP, (R)-Digm-BINAP, (R)-u-BINAP, (R)—H₈-BINAP. {(R)-BINAP is (R)-(+)-2,2′-bis(diphenylphosphino)-1,1′-binaphthyl; (R)-tol-BINAP is (R)-(+)-2,2′-bis(di-p-tolylphosphino)-1,1′-binaphthyl; (R)-Digm-BINAP is N,N′″-[[2,2′-bis(diphenylphosphino)-1,1′binaphthalene-6,6′diyl]bis(methylene)]diguanidine; (R)-u-BINAP is 2-[bis-(4-methoxy-3,5-dimethylphenyl)phosphino]-2′-diphenylphosphino-1,1′-binaphthyl; and, (R)—H₈-BINAP is (R)-2,2′-bis(diphenylphosphino)-5,6,7,8,5′,6′,7′,8′-octahydro-1,1′-binaphthyl.}

Suitable solvents include ethereal solvents in which the organic reaction components are sufficiently soluble, for example tetrahydrofuran, 2-methyl-tetrahydrofuran, dioxane, or methyl tert-butylether.

In a further aspect of the present invention there is provided a compound of the formula (II)

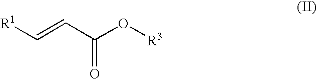

wherein R¹ is N—(SO₂CH₃)piperidin-4-yl and R³ is hydrogen, ethyl, iso-propyl or tert-butyl.

Compounds of formula (II) and (III) can be prepared by using or adapting methods described in the literature or described herein.

The invention will now be illustrated by the following non-limiting Examples in which, unless stated otherwise:

(i) temperatures are given in degrees Celsius (° C.); operations were carried out at room or ambient temperature, that is, at a temperature in the range of 18-25° C.;
(ii) evaporation of solvent was carried out using a rotary evaporator under reduced pressure (600-4000 Pascals; 4.5-30 mm Hg) with a bath temperature of up to 60° C.;
(iii) chromatography unless otherwise stated means flash chromatography on silica gel; thin layer chromatography (TLC) was carried out on silica gel plates;
(iv) in general, the course of reactions was followed by LC or GC and reaction times are given for illustration only;
(v) yields, when given, are for illustration only and are not necessarily those which can be obtained by diligent process development; preparations were repeated if more material was required;
(vi) when given, ¹H NMR data is quoted and is in the form of delta values for major diagnostic protons, given in parts per million (ppm) relative to tetramethylsilane (TMS) as an internal standard, determined at the specified frequency in the specified deuterated solvent; coupling constants (J) are given in Hz;
(vii) chemical symbols have their usual meanings; SI units and symbols are used; and,
(viii) the following abbreviations are used:

THF Tetrahydrofuran;

DCM Dichloromethane

DIPE Di-iso-propyl ether

DIBAL Di-iso-butylaluminium hydride

DMSO Dimethylsulfoxide

IPA Iso-propanol

R-BINAP (R)-2,2′-Bis(diphenylphosphino)-1,1′-binaphthyl

TPAP Tetrapropylammonium perruthenate

Mol eq Molar equivalents

Rel vol Relative volume

MTBE Methyl tert-butylether

### Preparation 1

### Preparation of 1-methanesulfonyl-4-(ethoxycarbonyl)-piperidine

Ethyl isonipecotate (1 mol eq) was charged to a reaction vessel followed by a line wash of DCM (1 rel vol). Triethylamine (1 mol eq) was charged to the vessel followed by a line wash of DCM (1 rel vol). DCM (5 rel vol) was charged to the vessel and the reaction mixture cooled to between 0 and 5° C. A solution of methane sulfonyl chloride (1 mol eq) in DCM (2 rel vol) followed by a line wash of DCM (1 rel vol) was added to the vessel maintaining the temperature between 1 and 10° C. The reaction mixture was stirred at between 0 and 10° C. until the reaction was complete. Purified water (5 rel vol) was charged to the reaction mixture and stirred for 15 minutes at between 5 and 10° C. The resulting phases were separated and the organic phase was concentrated to approximately 4.5 rel vol by atmospheric distillation. The concentrate was clarified, and then DIPE (10 rel vol) was added and the reaction concentrated again to approximately 4.5 rel vols by reduced pressure distillation. Another portion of DIPE (10 rel vol) was added and the resulting suspension was stirred at ambient temperature for at least 60 minutes. The solid was isolated by filtration, washed with DIPE (2 rel vols) and then dried at ambient temperature to give the sub-titled compound in approximately 93% yield.

¹H NMR (400 MHz, DMSO-d⁶) δ 4.05 (q, J=7.1 Hz, 2H), 3.46 (d, J=12.0 Hz, 2H), 2.81 (s, 3H), 2.76 (t, J=11.5 Hz, 2H), 2.48-2.38 (m, 1H), 1.90 (d, J=13.3 Hz, 2H), 1.56 (dd, J=35.4, 3.5 Hz, 2H), 1.16 (t, J=7.2 Hz, 3H).

### Preparation 2

### Preparation of (1-methanesulfonylpiperidin-4-yl)methanol

1-Methanesulfonyl-4-(ethoxycarbonyl)-piperidine (1 mol eq) was charged to a reaction vessel followed by a line wash of THF (6 rel vols). The reaction mixture was cooled to between 0 and 10° C. A solution of lithium aluminium hydride (1M in THF, 0.75 mol eq) followed by a line wash of THF (1 rel vol) was added to the vessel, keeping the temperature between 0° and 20° C., and then the reaction mixture was warmed to ambient temperature and stirred until the reaction was complete. The reaction mixture was cooled to between 0 and 2° C. Purified water (1 rel vol) was then charged to the vessel maintaining the temperature between 0° to 10° C. The pH of the reaction was adjusted to <2 by charging 5M HCl, maintaining the temperature between 0 and 10° C. The reaction mixture was warmed to room temperature, stirred for at least 15 minutes and then the phases separated. DCM (5 rel vol) was charged to the aqueous phase, stirred for at least 15 minutes and the phases separated. The first organic (THF) phase was concentrated to approximately 3.5 rel vols by vacuum distillation at 40° C. The second organic (DCM) phase was added to the concentrate, the phases separated and the organic phase concentrated to approximately 3.5 rel vol by atmospheric distillation. DIPE (10 rel vol) was added to the residue from the distillation at 40 to 45° C. After concentration to approximately 5 rel vol by vacuum distillation more DIPE (5 rel vol) was added and the resulting slurry cooled to ambient temperature and stirred for approximately 60 minutes. The sub-titled compound was isolated by filtration, washed with DIPE (2×1 rel vol) and dried at ambient temperature to give the sub-titled compound in approximately 87% yield

¹H NMR (400 MHz, CDCl₃) δ 3.84 (dd, J=9.6, 2.2 Hz, 2H), 3.54 (d, J=4.9 Hz, 2H), 2.78 (s, 3H), 2.67 (t, J=12.0 Hz, 2H), 1.70-1.56 (m, 2H), 1.54 (s, 1H), 1.36 (qd, J=12.5, 4.2 Hz, 2H).

### Preparation 3

### Preparation of (1-methanesulfonylpiperidin-4-yl)methanol

### Method A

(1-Methanesulfonylpiperidin-4-yl)methanol (1 mol eq) was dissolved in DCM (5 rel vol) in a reaction vessel followed by a line wash of DCM (1.2 rel vol). Pyridinium chlorochromate (1 mol eq) as a slurry in DCM (10 rel vol) was added followed by DCM (5×1.2 rel vol) as line washes. The reaction mixture was stirred overnight at ambient temperature, after which water (18.3 rel vol) was added and the phases separated and the DCM phase passed through a short “pad” of silica eluting with EtOAc. The solvent was evaporated from the filtrate to leave the sub-titled compound as a solid in approximately 40% yield.

### Method B

(1-Methanesulfonylpiperidin-4-yl)methanol (1 mol eq) and molecular sieves (2.5 weight eq) and TPAP (0.05 mol eq) were charged to a reaction vessel with DCM (30 rel vols). N-Methyl-morpholine N-oxide (1.5 mol eq) was dissolved in DCM (5 rel vols) in a separate vessel and added to the first vessel, keeping the temperature below 24° C. Once the reaction had reached completion the reaction mixture was filtered through celite and the solvent evaporated from the filtrate in vacuo to leave the sub-titled as a white solid in approximately 40% yield.

### Method C

1-Methanesulfonyl-4-(ethoxycarbonyl)-piperidine (1 mol eq) was weighed into a reaction vessel with DCM (16 rel vol) and cooled to −77° C. DIBAL (1M in THF, 1.5 mol eq) was added slowly, keeping the temperature of the reaction below −75° C. After 3 hours another charge of DIBAL solution (1.5 mol eq) was added at low temperature. Once the reaction had reached completion the reaction mixture was quenched with ammonium chloride solution (20% w/w, 2 rel vol), keeping the temperature below −67° C. After stirring at this temperature for 30 minutes, HCl (2M, 2 rel vol) was added, again keeping the temperature below −68° C. The resulting mixture was allowed to warm to ambient temperature overnight to give a white slurry. Water, HCl (5M) and brine were added until the precipitate dissolved. The layers were separated and solvent was removed from the organic layer by evaporation in vacuo to give the sub-titled compound in approximately 65% yield (contaminated with 1-methanesulfonylpiperidin-4-yl)methanol).

### Method D

A solution of DCM (5 rel vol) and oxalyl chloride (3 mol eq) was cooled to below −70° C. In a separate vessel, DCM (2 rel vol) and DMSO (6 mol eq) were mixed before addition to the oxalyl chloride solution via a syringe, keeping the temperature below −64° C. during the addition. After stirring for 10 minutes a solution of (1-methanesulfonylpiperidin-4-yl)methanol (1 mol eq) in DCM (5 rel vol) and DMSO (0.5 rel vol) was added, keeping the temperature below −60° C. during the addition. The reaction mixture was held at −70° C. for 40 minutes before adding triethylamine (7.5 mol eq) slowly via a syringe. The reaction mixture was allowed to warm to room temperature overnight. HCl (2M, 5 rel vol) was added while cooling the reaction in an ice-water bath. DCM (5 rel vol) was added before separating the layers and washing the DCM layer with: HCl (2M, 5 rel vol); then sodium bicarbonate solution (saturated, 5 rel vol); and finally brine (5 rel vol). The organic solvent was removed from the organic phase in vacuo to leave the sub-titled in approximately 75% yield.

¹H NMR (400 MHz, CDCl₃) δ 9.69 (s, 1H), 3.68-3.54 (m, 2H), 2.96 (ddd, J=12.3, 9.7, 2.8 Hz, 2H), 2.78 (s, 3H), 2.43 (dquintet, J=9.5, 4.7 Hz, 1H), 2.10-2.00 (m, 2H), 1.81 (dtd, J=13.8, 9.8, 3.9 Hz, 2H).

### Preparation 4

### Preparation of Iso-Propyl Malonic Acid

Meldrum's acid (1 mol eq) was charged to a reaction vessel followed by toluene (5 rel vol) and IPA (0.59 rel vol). The reaction mixture was heated to between 85 and 90° C. until the reaction was complete. The reaction mixture was then cooled to ambient temperature and transferred to a suitable storage container, washing the vessel with toluene (1 rel vol) and adding this wash to the solution of the sub-titled compound.

### Preparation 5

### Preparation of Iso-Propyl 3-(1-methanesulfonylpiperidin-4-yl)propenoate

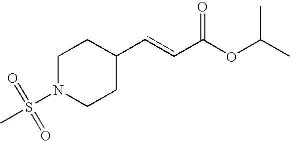

(1-Methanesulfonylpiperidin-4-yl)methanal (1 mol eq) was charged to a reaction vessel followed by a line wash of toluene (11 rel vol). Piperidine (0.1 mol eq) was charged to the vessel followed by a line wash of toluene (0.5 rel vol), and the reaction mixture heated to between 85 and 95° C. A solution of the iso-propyl malonic acid (1.25 mol eq) in toluene (prepared as described above) was added in 10 approximately equal portions over 6 to 8 hours and the reaction mixture was stirred at to between 85 and 95° C. until it reached completion. The reaction mixture was then cooled to between 40 and 50° C. and HCl (0.5M, 3 rel vol) was added to the reaction maintaining the temperature between 40 and 50° C. After stirring for at least 15 minutes the phases were separated. Sodium bicarbonate (0.5M, 3 rel vol) was added to the organic phase, still maintaining the temperature between 40 and 50° C. The 2-phase mixture was stirred for at least 15 minutes before separating the phases and washing the organic phase with water (3 rel vol). The organic phase was then concentrated to approximately 16 rel vols by vacuum distillation at between 40 and 50° C. Toluene (3.5 rel vol) was charged, the solution clarified at between 40 and 50° C. and then concentrated to approximately 7 rel vol by vacuum distillation. The mixture was then cooled to between 0 and 10° C. and stirred for at least 60 minutes at this temperature before isolating the sub-titled compound by filtration and washing the residue with toluene (2 rel vol) at between 0 and 10° C. The solid was dried to leave the sub-titled compound in approximately 59% yield.

¹H NMR (400 MHz, CDCl₃) δ 6.87 (dd, J=15.8, 6.5 Hz, 1H), 5.81 (dd, J=15.8, 0.9 Hz, 1H), 5.07 (quintet, J=6.2 Hz, 1H), 3.82 (d, J=12.0 Hz, 2H), 2.79 (s, 3H), 2.74 (td, J=12.0, 2.4 Hz, 2H), 2.36-2.17 (m, 1H), 1.95-1.80 (m, 2H), 1.57 (ddd, J=24.9, 11.7, 4.0 Hz, 2H), 1.27 (d, J=6.4 Hz, 6H).

Using similar methodology the following compounds were made:

### Ethyl 3-(1-methanesulfonylpiperidin-4-yl)propenoate

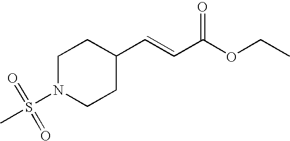

¹H NMR (400 MHz, CDCl₃) 1.30 (3H, t, J=7), 1.57 (2H, qd, J=12 and 4), 1.88 (2H, d, J=13.5), 2.27 (1H, m), 2.74 (2H, td, J=11.5 and 2), 2.79 (3H, s), 3.82 (2H, m), 4.20 (2H, q, J=7), 5.83 (2H, d, J=16), 6.89 (1H, dd, J=16 and 6.5).

### Tert-Butyl 3-(1-methanesulfonylpiperidin-4-yl)propenoate

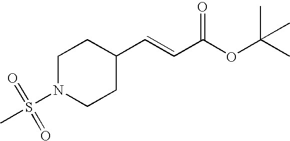

¹H NMR (400 MHz, CDCl₃) 1.45-1.62 (2H, m), 1.49 (9H, s), 1.87 (2H, m), 2.24 (1H, m), 2.74 (2H, td, J=12 and 2.5), 2.79 (3H, s), 3.81 (2H, m), 7.75 (1H, dd, J=15.5, 1.5), 6.79 (1H, dd, J=15.5 and 6.5).

### Preparation 6

### Preparation of 3-(1-methanesulfonylpiperidin-4-yl)propenoic Acid

(1-Methanesulfonylpiperidin-4-yl)methanal (1 mol eq) was charged to a reaction vessel with toluene (0.35 rel vol), pyridine (1.2 rel vol) and piperidine (0.014 mol eq). Malonic acid (1.12 mol eq) was added as a solid and the reaction mixture heated to 87° C. for 1 hour followed by 106° C. for 45 minutes. After another charge of piperidine (0.0104 mol eq) and malonic acid (0.7 mol eq) and further stirring at 106° C. the reaction had reached completion. The reaction mixture was cooled to 50° C. and MTBE (3.3 rel vol) added causing precipitation of the sub-titled compound, which was isolated by filtration under suction. The residue was washed on the filter with MTBE (2×3.3 rel vol) and dried under vacuum overnight to give the sub-titled compound in approximately 92% yield.

¹H NMR (400 MHz, DMSO-d⁶) δ 6.78 (dd, J=15.7, 6.5 Hz, 1H), 5.75 (dd, J=15.7, 1.2 Hz, 1H), 3.54 (d, J=12.0 Hz, 2H), 2.82 (s, 3H), 2.72 (td, J=11.9, 2.3 Hz, 2H), 2.26 (dt, J=7.2, 3.6 Hz, 1H), 1.78 (d, J=11.0 Hz, 2H), 1.37 (qd, J=12.2, 3.6 Hz, 2H).

### Preparation 7

### Preparation of 2-(3,5-Difluorophenyl)-5,5-dimethyl-1,3,2-dioxaborinane

(This compound is sold commercially with the name 3,5-difluorobenzeneboronic acid neopentyl glycol cyclic ester.)

Toluene (17 rel vols), 3,5-difluorophenyl boronic acid (1 mol eq) and neopentyl glycol (1.25 eq) were charged to a reaction vessel and heated to reflux (118° C.) under Dean-Stark conditions for 2 hours. The volume of the reaction was then reduced to 4 rel vols and then cooled to 50° C. Water (3 rel vols) was added and the phases separated. Brine (3 rel vols) was added and the phases separated. The solvent was removed from the organic layer by distillation in vacuo to give the sub-titled in approximately 87% yield.

¹H NMR (400 MHz, CDCl₃) δ 7.27 (td, J=4.0, 2.1 Hz, 2H), 6.83 (dd, J=11.4, 6.5 Hz, 1H), 3.76 (s, 4H), 1.02 (s, 6H).

### Preparation 8

### Preparation of N-(1-{(3R)-3-(3,5-difluorophenyl)-3-[1-(methylsulfonyl)piperidin-4-yl]propyl}piperidin-4-yl)-N-ethyl-2-[4-(methylsulfonyl)phenyl]acetamide

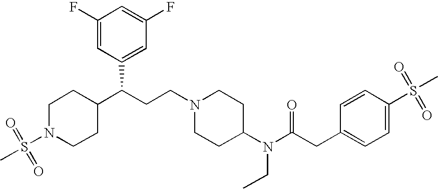

Iso-Propyl (3R)-3-(3,5-difluorophenyl)-3-[1-(methylsulfonyl)piperidin-4-yl]propanoate (1 mol eq) was charged to a reaction vessel followed by THF (5 rel vols) and the resulting mixture was cooled to −10° C. DIBAL (1M in THF, 3.4 mmol eq) was added keeping the temperature between −5 and −10° C. during the addition. After the reaction had reached completion the reaction mixture was quenched by adding it to a mixture of HCl (5M, 6.2 mol eq) and NaCl solution (10% w/v, 3.7 rel vol) at 35° C. The resulting mixture was cooled to 20° C. and the phases separated, washing the organic phase with NaCl solution (10% w/v, 3.6 rel vol). The volume of the organic phase was reduced to 4 rel vol by distillation at atmospheric pressure and acetonitrile (8 rel vol) was then added. This was repeated 3 times. The solution was then cooled to 0° C. and tosyl chloride (1.25 mol eq) and trimethylamine hydrochloride (0.095 mol eq) were added followed by acetonitrile (2.1 rel vol). A mixture of triethylamine (1.8 mol eq) in acetonitrile (0.75 rel vol) was prepared in a separate vessel and added to the reaction vessel, keeping the temperature between 0 and 5° C. during the addition. After the reaction reached completion, water (0.14 rel vol) was added followed by HCl (5M, 0.5 mol eq) and KCl solution (10% w/v, 4 rel vol). The phases were separated and the organic phase washed with KCl solution (10% w/v, 3 rel vol). Potassium carbonate (3.5 mol eq) and N-ethyl-2-[4-(methylsulfonyl)phenyl]-N-piperidin-4-ylacetamide (0.95 mol eq) were charged to the reaction mixture followed by acetonitrile (9.7 rel vol). The reaction mixture was heated to 75° C. and held for 20 hours, after which water (7.5 rel vol) was added and the mixture and the temperature held at 50° C. until all solids had dissolved. The reaction mixture was then cooled to ambient temperature and the phases separated, retaining the organic phase. Acetonitrile was added to return the volume of the reaction mixture to around 13.8 rel vol. KOH (0.5% in 10% w/v KCl solution, 8.4 rel vols) was added and the phases separated, followed by another charge of acetonitrile to maintain the organic phase at around 14 rel vol. After a second KOH wash (8.4 rel vols) the organic phase was reduced in volume to 4 rel vols by atmospheric distillation. Acetonitrile was added to give a volume of 7 rel vols which was distilled to 4 rel vols again. After diluting to about 8 rel vols with acetonitrile, the reaction was heated to 75° C. and succinic acid (0.86 mol eq) was added, followed by acetonitrile (3 rel vols). The mixture was screened into a clean vessel and cooled to 60° C. before adding a seed (which was made by withdrawing a small portion of the reaction and cooling to obtain a solid, before returning this solid to the reaction). The reaction was then held for 4 hours at 60° C. and then cooled to 15° C. The product was isolated by filtration under suction, washing the product with acetonitrile and drying in a vacuum oven to give the sub-titled compound in approximately 75% yield.

¹H NMR (400 MHz, DMSO-d⁶) δ 7.83 (d, J=8.2 Hz, 2H), 7.46 (dd, J=8.1, 4.0 Hz, 2H), 7.02 (t, J=9.2 Hz, 1H), 6.93 (d, J=6.7 Hz, 2H), 4.13-3.97 (m, 0.5H), 3.81 (d, J=15.9 Hz, 2H), 3.67-3.58 (m, 0.5H), 3.55 (d, J=11.8 Hz, 1H), 3.43 (d, J=37.2 Hz, 1H), 3.29 (d, J=6.9 Hz, 1H), 3.16-3.10 (m, 5H), 2.94-2.67 (m, 7H), 2.61 (t, J=11.3 Hz, 1H), 2.56-2.42 (m, 2H), 2.38 (s, 3H), 2.20-2.03 (m, 1H), 2.02-1.78 (m, 5H), 1.76-1.23 (m, 6H), 1.20-0.93 (m, 4H).

### EXAMPLE 1

### Preparation of Iso-Propyl (3R)-3-(3,5-difluorophenyl)-3-[1-(methylsulfonyl)piperidin-4-yl]propanoate (using 3,5-difluorophenylboronic Acid)

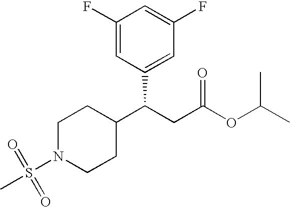

A catalyst solution was prepared by charging R-BINAP (0.045 mol eq) and bis(1,5-cyclooctadienerhodium chloride), (0.02 mol eq) to a vessel followed by THF (2.8 rel. vols). The mixture was stirred to achieve full dissolution.

To a larger reaction vessel was charged iso-propyl 3-(1-methanesulphonylpiperidin-4-yl)propenoate (1 mol eq), 3,5-difluorophenylboronic acid (1.35 mol eq) and potassium carbonate (1.35 mol eq). THF (7.8 rel vols) and IPA (1 mol eq) were then charged and the mixture was heated to 60° C. The catalyst solution was then added to this mixture, and a line wash of THF (1.4 rel vols) was used to facilitate this transfer. The resulting mixture was then held at 60° C. for 2 hours. The reaction mixture was cooled to room temperature a solution of L-cysteine (0.9 rel wt) in water (12 rel vols), was added. The resulting mixture was stirred at room temperature overnight. The phases were then separated and the organic portion was concentrated to a volume of 3.5 rel vols. IPA (10.5 rel vols) was then charged and the batch was then concentrated again to a volume of 3.5 rel vols. Further IPA (10.5 rel vols) was charged, and again the batch was concentrated to a volume of 3.5 rel vols. Finally a further 10.5 rel vols of IPA was charged, and the resulting mixture was held at 30-35° C. for 15-30 minutes, then heated to 70° C. The mixture was then filtered into a crystallisation vessel. A line wash of IPA (1.5 rel vols) was used to facilitate transfer.

Around 1% of the crystallisation solution was removed to provide a seed sample. This crystallised upon standing.

The crystallisation solution was cooled to 50° C., and then was cooled at 12° C./hour to 20° C. The seed was added when the crystallisation solution was at 40° C. The crystallisation solution was held at room temperature overnight.

The crystallised product was isolated by suction filtration. The resulting cake was washed with IPA (3.5 rel vols). The washed cake was then dried to constant mass in a vacuum oven at 50° C. to afford the sub-titled compound in 75% yield.

¹H NMR (400 MHz, DMSO-d⁶) 0.96 (3H, d, J=6), 1.02 (3H, d, J=6), 1.10 (1H, qd, J=12.5 and 4), 1.18 (1H, qd, J=12.5 and 4), 1.33 (1H, d, J=12.5), 1.60 (1H, m), 1.88 (1H, d, J=12.5), 2.49-2.66 (3H, m), 2.80 (1H, dd, J=15 and 5), 2.81 (3H, s), 2.91 (1H, m), 3.46 (1H, d, J=12), 3.57 (1H, d, J=12), 4.71 (1H, septet, J=6), 6.98 (2H, dd, J=8 and 1.5), 7.05 (1H, tt, J=9.5 and 1.5).

Using similar methodology the following compounds were made:

### 1. Iso-Propyl (3R)-3-(3,5-difluorophenyl)-3-[4-(methylsulfonyl)phenyl]propanoate

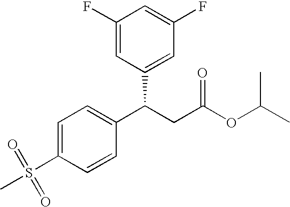

¹H NMR (400 MHz, CDCl₃) 1.11 (3H, d, J=6), 1.12 (3H, d, J=6), 3.01-3.05 (5H, m), 4.62 (1H, t, J=8), 4.93 (1H, septet, J=6), 6.68 (1H, tt, J=8.2 and 2), 6.75-6.80 (2H, m), 7.45 (2H, d, J=8), 7.90 (2H, d, J=8).

### 2. Iso-Propyl (3R)-3-(3,5-difluorophenyl)-3-[4-(methylthio)phenyl]propanoate

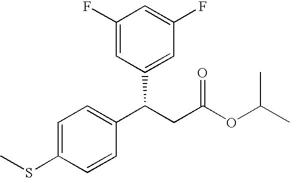

¹H NMR (400 MHz, CDCl₃) 1.10 (6H, d, J=6), 2.45 (3H, s), 2.94 (2H, d, J=8), 4.46 (1H, t, J=8), 4.92 (1H, septet, J=6), 6.63 (1H, tt, J=9 and 2), 6.72-6.77 (2H, m), 7.12 (2H, d, J=8), 7.19 (2H, d, J=8).

### 3. Iso-Propyl (3R)-3-(3,5-difluorophenyl)-3-[4-nitrophenyl]propanoate

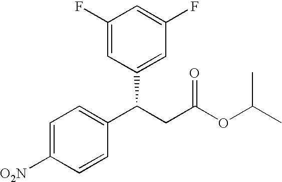

¹H NMR (400 MHz, CDCl₃) 1.12 (3H, d, J=6), 1.13 (3H, d, J=6), 3.01 (1H, d, J=8), 4.62 (1H, t, J=8), 4.92 (1H, septet, J=6), 6.68 (1H, tt, J=9 and 2.5), 6.72-6.77 (2H, m), 7.39 (2H, d, J=8.5), 8.18 (2H, d, J=8.5).

### 4. Iso-Propyl (3R)-3-(3,5-difluorophenyl)-3-phenylpropanoate

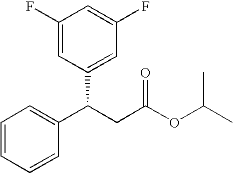

¹H NMR (400 MHz, CDCl₃) 1.09 (3H, d, J=6.5), 1.10 (3H, d, J=6.5), 2.97 (2H, d, J=8), 4.50 (1H, t, J=8), 4.92 (1H, sextet, J=6.5), 6.62 (1H, tt, J=9 and 2.5), 6.73-6.80 (2H, m), 7.18-7.32 (5H, m).

### 5. Iso-Propyl (3R)-3-(3,5-difluorophenyl)-3-(4-methoxyphenyl)propanoate

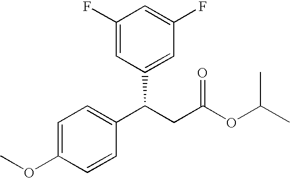

¹H NMR (400 MHz, CDCl₃) 1.10 (6H, d, J=6.5), 2.94 (2H, d, J=8), 3.77 (3H, s), 4.92 (1H, septet, J=6.5), 6.62 (1H, tt, J=9.5 and 2.5), 6.72-6.77 (2H, m), 6.83 (2H, d, J=8.5), 7.12 (2H, d, J=8.5).

### EXAMPLE 2

### Preparation of Iso-Propyl (3R)-3-(3,5-difluorophenyl)-3-[1-(methylsulfonyl)piperidin-4-yl]propanoate (Using 2-(3,5-difluorophenyl)-5,5-dimethyl-1,3,2-dioxaborinane)

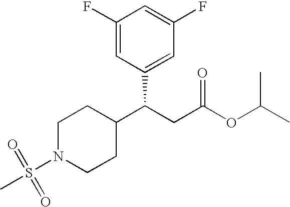

A catalyst solution was prepared by charging R-BINAP (0.035 mol eq) and bis(1,5-cyclooctadienerhodium chloride), (0.015 mol eq) to a vessel followed by THF (2.0 rel. vols). The mixture was stirred to achieve fall dissolution.

To a larger reaction vessel was charged iso-propyl 3-(1-methanesulfonylpiperidin-4-yl)propenoate (1 mol eq), 2-(3,5-difluorophenyl)-5,5-dimethyl-1,3,2-dioxaborinane (1.5 mol eq) and potassium carbonate (0.2 mol eq). THF (10 rel vols) and IPA (1.1 mol eq) were then charged and the mixture was heated to 60° C. The catalyst solution was then added to this mixture, and the reaction mixture was held at 60-66° C. for 2 hours. The crude reaction mixture was concentrated in vacuo. The residue was largely dissolved into MTBE, and this solution was filtered through a pad of silica. The resulting solution was concentrated in vacuo and was triturated using iso-hexane and MTBE. The resulting solid was collected by filtration, and dried overnight in a vacuum oven at 40° C. The title compound was afforded in 67% yield.

Using similar methodology the following compounds were made:

### 1. Ethyl (3R)-3-(3,5-difluorophenyl)-3-[1-(methylsulfonyl)piperidin-4-yl]propanoate

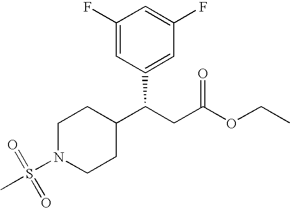

¹H NMR (400 MHz, CDCl₃) 1.12 (3H, t, J=7), 1.24 (1H, qd, J=12.5 and 4.5), 1.39 (1H, qd, J=12.5 and 4.5), 1.50 (1H, dt, J=14 and 1.5), 1.59 (1H, m), 1.89 (1H, dt, J=12.5 and 2.5), 2.49-2.58 (2H, m), 2.63 (1H, td, J=9.5 and 2.5), 2.75 (3H, s), 2.76 (1H, dd, J=15 and 5), 2.96 (1H, td, J=9 and 5), 3.74 (1H, m), 3.85 (1H, m), 4.02 (2H, q, J=7), 6.65-6.72 (3H, m).

### 2. Tert-Butyl (3R)-3-(3,5-difluorophenyl)-3-[1-(methylsulfonyl)piperidin-4-yl]propanoate

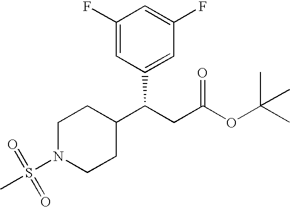

¹H NMR (400 MHz, CDCl₃) 1.20-1.60 (4H, m), 1.25 (9H, s), 1.88 (1H, m), 2.40-2.75 (4H, m), 2.75 (3H, s), 2.90 (1H, m), 3.74 (1H, m), 3.85 (1H, m), 6.64-6.72 (3H, m).

### 3. Benzyl 4-[(1R)-1-(3,5-difluorophenyl)-3-isopropoxy-3-oxopropyl]piperidine-1-carboxylate

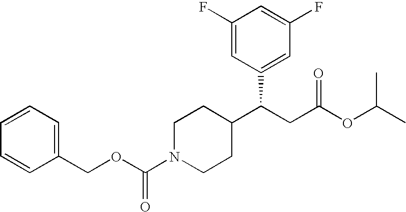

The e.e. in this instance was determined to be 78-80%.

¹H NMR (400 MHz, CDCl₃) 1.04 (3H, d, J=6), 1.10 (3H, d, J=6), 1.10-1.25 (2H, m), 1.38 (1H, br m), 1.62 (1H, br m), 1.76 (1H, br m), 2.49 (1H, dd, J=15 and 9.5), 2.64 (1H, br m), 2.73 (2H, dd, J=15 and 5.5), 2.91 (1H, m), 4.12-4.32 (2H, br m), 4.86 (1H, septet, J=6), 5.10 (2H, s), 6.62-6.71 (3H, m), 7.26-7.38 (5H, m).

1. A process for preparing a compound of formula (I): 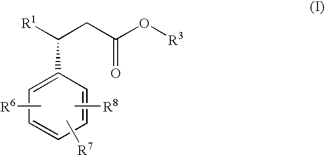
wherein R¹ is N-substituted piperidin-4-yl or optionally substituted phenyl; R³ is C₁₋₆ alkyl, optionally substituted phenyl or optionally substituted phenyl(C₁₋₄ alkyl); R⁶ is fluoro; and R⁷ and R⁸ are, independently, hydrogen or fluoro; the process comprising reacting a compound of formula (II): 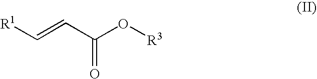
with 1 to 2 (for example 1 to 1.5) molar equivalents of a fluorinated boron species of formula (III): 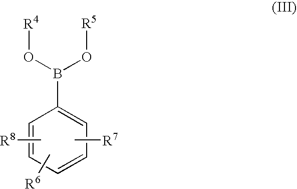
wherein R⁴ and R⁵ are, independently, hydrogen, C₁₋₆ alkyl, phenyl or phenyl(C₁₋₄ alkyl); or R⁴ and R⁵ join to form a ring;
in the presence of:
0.8 to 1.5 molar equivalents of an alcohol;
a rhodium (I) pre-catalyst species;
a suitable ligand that binds to the rhodium (I) pre-catalyst species to form a catalyst complex;
a base; and,
a suitable solvent;
the process being carried out at a temperature in the range 40 to 110° C.

2. A process for preparing a compound of formula (I) as claimed in claim 1 wherein
R¹ is phenyl optionally substituted by halo, S(O)₂(C₁₋₄ alkyl), S(O)₂(C₁₋₄ haloalkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C₁₋₄ haloalkyl, C₁₋₄ haloalkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl) or C(O)(C₁₋₄ haloalkyl).

3. A process for preparing a compound of formula (I) as claimed in claim 1 wherein N-substituted piperidin-4-yl is piperidin-4-yl with C₁₋₄ alkyl, S(O)₂(C₁₋₄ alkyl), S(O)₂(C₁₋₄ haloalkyl), C(O)(C₁₋₄ alkyl) or C(O)(C₁₋₄ haloalkyl) on the ring nitrogen.

4. A process for preparing a compound of formula (I) as claimed in any preceding claim wherein R³ is ethyl, iso-propyl or tert-butyl.

5. A process for preparing a compound of formula (I) as claimed in any preceding claim wherein R⁶ is 3-fluoro; and R⁷ and R⁸ are, independently, hydrogen or fluoro.

6. A process for preparing a compound of formula (I) as claimed in any preceding claim wherein the alcohol is a C₁₋₁₀ aliphatic straight or branched chain acyclic alcohol or a C₃₋₁₀ cyclic alcohol.

7. A process for preparing a compound of formula (I) as claimed in any preceding claim wherein the base is a phosphate, carbonate or bicarbonate of an alkali metal or alkaline earth metal.

8. A process for preparing a compound of formula (I) as claimed in any preceding claim wherein the rhodium (I) pre-catalyst species is acetylacetobis[ethylene]rhodium (I), [Rh(COD)Cl]₂ or [Rh(COD)(MeCN)₂]BF₄.

9. A compound of the formula (II) 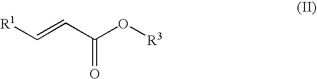
wherein R¹ is N—(SO₂CH₃)piperidin-4-yl and R³ is hydrogen, ethyl, iso-propyl or tert-butyl.
